(19) United States
(12) Patent Application Publication
(10) Pub. No.: US 2013/0103105 A1
(43) Pub. Date: Apr. 25, 2013

(54) DEVICE FOR FACILITATING THE APPLICATION OF A FIXING PLATE TO THE RELATIVE SCREW FOR THE MINIMALLY INVASIVE STABILIZATION OF PERTROCHANTERIC FEMORAL FRACTURES WITH SLIDING SCREW-PLATE SYSTEMS

(71) Applicant: Ferdinando Del Prete, Firenze (IT)

(72) Inventor: Ferdinando Del Prete, Firenze (IT)

(21) Appl. No.: 13/806,418

(22) PCT Filed: Jun. 30, 2011
(86) PCT No.: PCT/IB2011/052887

§ 371 Date: Dec. 21, 2012

(30) Foreign Application Priority Data
Jul. 1, 2010 (IT) PI2010A000081

(51) Int. Cl. A61B 17/80

(52) U.S. Cl. CPC A61B 17/808
USPC 606/86 B

(57) ABSTRACT

The present invention concerns a device (1) for allowing the application of a fixing plate (20) on a compression screw (10) in a bone fracture, in particular a femoral fracture. In accordance with the invention, a generally long-shaped element (1) is included, provided on one of its ends (2) with fixing means (4, 16, 6) for rendering applicable the device to the end (13) of the compression screw (10). On the opposite side, a bevel (3) is included, configured in such a way as to realize a stress for the insertion in use of the plate when the channel of the plate is not in axis with the screw.

### TECHNICAL FIELD

The present invention refers to the technical field relative to the orthopaedic surgical devices.

In particular, the invention refers to an innovative device that facilitates, during a surgical intervention, the insertion of a fixing plate on the relative compression screw used for stabilizing the femoral fracture.

### BACKGROUND ART

It is known that the proximal femoral fracture is something that frequently occurs to old people, above all if affected by osteoporosis. Such fractures, if not adequately treated through a proper surgical intervention, produce a mortality rate variable from 30% to 50% of the injured people. It is in fact frequent the case in which such patients are in a comorbidity condition, that is affected at the same time by other illnesses generally due to old age, and have therefore a very limited recovery capacity. In this case, the additional trauma of a prolonged immobility in a strange environment facilitates the spreading of such illnesses, inevitably conducting to death. In virtue of what has been said, it is clear that the fracture in an old patient represents a challenge for the orthopaedic surgeon that has the aim of bringing back with the intervention the level of autonomy and functionality present before the trauma in a short time, above all in the case of patients with said comorbidity, in such a way as to reduce to the minimum the post-intervention mortality rate.

In the specific case, proximal femoral fractures can be of two main types: femur neck or trochanteric. The first ones are treated with the intervention of prosthesis, while the second ones have the indication to the fixation with synthesis means.

It is precisely in the case of trochanteric fractures that, considering the actual state of the art, no synthesis means are present configured to allow an application of the same according to a mini-invasive technique, therefore suitable for optimizing the post-intervention recovery.

The sliding screw-plate represents one of the said stabilization means most commonly used for the synthesis of such types of fractures. This type of synthesis means, as schematically represented in FIG. 1, consists of a screw 10 which is inserted from the lateral face of the femur in combination with a fixing plate 20 provided with a sliding channel 21. In this way, a compression takes place at fracture level, maintaining well welded the fractured part and distributing the load on the healthy part of the bone. Thanks to such a system of synthesis, the patient has a rapid recovery and a less painful confinement in bed.

In accordance with the background art, therefore, FIG. 1 shows the compression screw 10 comprising a head 11 threaded and a cylindrical body 12 provided with two milled faces, in such a way as to avoid any relative rotation with respect to the plate when the plate 20 is applied to the screw through the channel 21. To that aim, the channel 21 is also internally milled in such a way as to hold inside the screw without any relative rotation between them. Moreover, the tolerance between the channel and the screw is very tight so as to allow the sliding avoiding at the same time backlashes that could engage dangerous torsions and loss of the reduction of the fracture. The screw thread 11 can be of different forms and types, for example of the so-called “spiral blade” type.

The screw 10 is also canulated, which means that it includes a tunnel that runs axially along all the length in the bone channel, applying it on the guide wire from the threaded head 11. The guide wire is used during the phase of engagement in order to verify the correct axis of insertion of the screw.

The attachment 13, opposed to the screw thread, is configured to be connected to a screwing/insertion device 30 comprising a long rod. The length of such rod must be such as to exit in abundance from the skin incision on the thigh 50 of the patient to allow the surgeon the precise insertion of the channel of the plate on the screw, maintaining the angle, the fixing on the milled faces and the axis plate—femur (see FIG. 2, for example). The diameter of the screw thread 11 is greater with respect to the body 12 of the screw in such a way that, once the screw is inserted, the screw thread sets on the bone, leaving a backlash between the body of the screw and the channel 40 of insertion obtained in the bone (see figure again). Such a backlash allows the insertion of the channel 21 into the channel 40 through its sliding on the screw. The channel 21 results comprised between the screw and the walls of the channel 40.

As shown in FIG. 2, in fact, once the screw is inserted, the insertion of the sliding channel 21 takes place on the rod 30 from the part exiting from the thigh and that serves as guide (FIG. 2 highlights the insertion angle α of the rod 30).

The rod 30 has a length even superior to the 15 centimetres and the plate can slide on the rod up to when the channel reaches the body of the screw and the plate is in direct contrast with the bone (see plate in thin dotted line in FIG. 2). The plate includes also a plurality of holes 22 along its body through which to apply the fixing screws that fix the plate to the bone (see horizontal axis in dotted line in FIG. 2).

That being stated, it is clear that such a device obliges the surgeon to practise a particularly importation incision in order to insert the plate correctly, above all in the case of obese patients or patients with significant muscular masses.

The rod 30, in fact, obliges the plate to a rigid translation motion and does not give the surgeon the possibility of either moving it freely, let alone of rotating it with respect to the rod itself by means of the milling. In this case, to proceed with the correct insertion, the surgeon is obliges to make the plate slide up to when it reaches the thigh 50. Once the thigh 50 is reached, having the plate a length even superior to the 15 centimetres, to proceed with the insertion it is necessary to practise an incision longer than the plate itself (the incision starts from the engagement point of the rod 30 up to covering at least the entire length of the plate itself). The final result will be therefore of particularly long incisions (also superior to the 20 centimetres), which can engage additional problems, such as important haematic problems, long recovery periods, and the presence of visible and permanent scars.

U.S. Pat. No. 3,791,380 describes a similar technique wherein a plate (k) is fixed to the bone of the fractured femur (u) through a tubular element provided on one of its ends with a rod (f) threaded and of quadrangular section and on the opposite end with an element (c) provided with four hooks (b′), extractable and retractable. The hooks realize the grasping in the bone that consolidates better the fracture, while the rod f serves for the insertion and the sliding of the plate (k).

Nevertheless, also in this case, exactly as discussed before, the length of the rod (f) is great and the end of the rod is conformed in such a way as to render really difficult the insertion of the channel of the plate on the rod itself until the axis of the channel is not in axis with the rod (f) itself. Also in this case, therefore, it is necessary to practise long incisions exactly as described before.

In U.S. Pat. No. 4,095,591 a similar plate and screw device is described that does not contribute in any way to the solution of said technical problem. The screw is screwed in the hole of the bone with the help of an insertion key of tubular shape into which the screw is engaged slidingly. The relative rotation between the two parts (screw-insertion key) is impeded by means of a key that engages in a corresponding longitudinal seat obtained in the body of the screw. Once the screwing has been realized, the key is taken away and the insertion of the plate through the sliding of its channel on the screw takes place. Also in this case, exactly as the preceding document, the part of projecting screw (length and conformation) is such as not to allow an insertion of the plate if it is not placed first with its channel in axis with the screw. This solution, exactly as the preceding one, requires the necessary realization of a significant cut.

In US2002/0143333 an alternative system is described for the recovery of a femoral fracture. In this case, the screw presents integrated a fixing plate that is screwed on a screw thread present along the body of the screw. Once the screw is inserted, the screwing of the plate along the body of the screw takes place in such a way that it is in contrast against the bone, screwing up the fracture. Such a system can be eventually integrated to a traditional plate that must be however arranged before the installation on the screw. In this regard, also in this case, such a device does not contribute in any way to solving the technical problem formulated in view of the fact that, being the plate fixed on the screw, it is anyway necessary to realize a long cut.

Alternatively, with the traditional plate-screw systems, it would be possible to avoid the use of the long rod 30. In this regard, the surgeon could practise a very small incision and proceed with the insertion of the plate starting from the end opposed to the channel and making it slide under the muscle up to when the channel is not in contrast against the head of the screw. In this way, a sort of mini-invasive technique would be practised. However, such an operation is not standardized and requires a particular manual skill, apart from being able to cause damages because of incorrect engagements. In fact, it is very difficult for the surgeon to manoeuvre the plate once it has been engaged under the muscle in order to align the channel to the screw and proceed with the insertion. In particular, it is very difficult for the surgeon to make the milling of the channel and the milling of the screw perfectly coincide in order to couple them correctly and then proceed with the alignment.

Other mini-invasive techniques are also very limitative and not adaptable to this specific applicative case of trochanteric fracture.

### DISCLOSURE OF INVENTION

It is therefore the aim of the present invention to provide a device for realizing the application of the plate to the compression screw that solves at least in part said drawbacks.

In particular, it is the aim of the present invention to provide a device that is capable of allowing an insertion of the plate on the screw without necessarily realizing a long incision, thus allowing the diffusion of a standardized mini-invasive application technique.

It is therefore the aim of the present invention to provide a device that substitutes efficiently the long rod of guide 30 used in the state of the art, thus allowing the complete manoeuvrability of the plate in order to reduce the incision and guarantee at the same time a precise and easy insertion.

These and other aims are therefore obtained through the realization of a device 1 for allowing the application of a plate 20, through its sliding channel 21, on a compression screw 10 in a bone fracture, in particular a femoral fracture, as per claim 1.

In accordance with the invention, the device (1) is in the shape of a pivot (1) and is provided with fixing means (4, 16, 6) to render it applicable to the end (13) of said compression screw (10).

The pivot (1) is provided, on the opposite part destined to the connection with the end (13), with at least one bevel (3) that sections the pivot askew according to a pre-determined β angle with respect to the longitudinal axis (14) in such a way as to at least reach the longitudinal axis (14) of the pivot (1) forming a flute-beak bevel.

The term oblique or askew in the present text indicates, in the broader sense of the term, a direction of bevel that forms a β angle between the surface 3 of the bevel and the longitudinal axis 14 (for example, that shown in FIG. 4) inferior to a right angle. In this regard, the cut is intended as oblique when the surface 3 is not substantially orthogonal to the longitudinal axis 14 of the pivot 1.

More preferably, the bevel is obtained with an net oblique cut through the entire thickness of the pivot.

In the present text, in broad sense, a flute-beak bevel is intended both in the case of surface 3 of the rectilinear type, and in the case of surface 3, concave or convex, but such as to at least reach the longitudinal axis 14 of the pivot.

Moreover, the pivot (1) is short, that is has an overall length comprised between about 0.5 cm and about 3 cm, and preferably, between about 0.5 cm and about 2 cm.

For example, particularly functional lengths can be those of 0.5 cm, 1 cm, 1.5 cm and 2 cm, 2.5 cm and 3 cm.

In this way, in use, said overall length of the pivot (1), in combination with said bevel (3) thus realized, allow to realize the insertion of the channel (21) on the compression screw (10), through the application of the pivot (1) on the screw (10), also when the channel (21) is not in axis with the screw (10).

The pivot in fact forms an extension of the screw, but of very contained length if compared to the traditional rods of the state of the art (as it has been said, they exceed also the 15 cm). Such length of the pivot allows to fit the end of the channel 21 on the pivot 1 once the plate 20 has been inserted and slided under the muscle through a small incision.

Initially, the channel, and therefore the plate, are arranged with a certain inclination with respect to the axis of the screw in the initial phase of insertion with the fitting of the channel that leans on the end of the flute-beak pivot. The flute-beak bevel 3 makes that a very slight pressure exerted by the surgeon is enough to make the channel slide on the bevel, causing a forwarding of the channel that brings it in axis with the insertion screw. In this way, in a quick and simple manner, the plate can be taken in position on the bone without having to operate long cuts and with extreme ease for the surgeon.

Advantageously, the pivot (1) can comprise two millings (15) opposed in such a way as to result insertable into the relative milled seat of the channel (21) of the plate (20) without relative rotation between the two parts.

Advantageously, the pivot (1) can comprise at least one pair of teeth (2′) for engaging each one of them into a respective complementary seat (13′) of the end (13) of the compression screw (10) in such a way as to oblige to realize an application of the pivot (1) to the compression screw (10) so that there is a continuity in the millings present on the compression screws (10) and on the pivot (1).

Advantageously, said β angle can be comprised within a range between 20° and 80° with respect to the longitudinal axis (14) of the pivot itself, and preferably within a range between 30° and 60°.

Advantageously, the pivot (1) is axially drilled and forms an axial channel (16).

Advantageously, the pivot (1) is substantially cylindrical.

Advantageously, the fixing means (4, 16, 6) can comprise:

A screw thread (6) placed in the final part of the axial channel (16) of the pivot (1);

A screw (4) placed into the channel (16) and having at least one threaded final part in such a way as to be able to screw it to the screw thread (6) of the channel (16), exiting from it at least in part to be able to engage into a corresponding screw thread arranged in axis at the end (13) of the compression screw (10).

In that case, advantageously, the screw (4) can include a first part (4′) non threaded, and a final part, threaded, the diameter of the non threaded part being inferior to the diameter of the seat (6) of the pivot into which the screw (4) is inserted and the length of the non threaded part (4′) being further greater than the length of the threaded part (6) in such a way that, when the threaded part of the screw (4) has exceeded the screw thread (6) of the axial channel (16), exiting from the pivot (1), the non-threaded part (4′) results sliding with respect to the threaded part (6) of the channel (16) of a quantity equivalent to the excess of length between the non threaded part (4′) and the length of the threaded part (6) in such a way as to allow to continue the screwing of the screw (4) in the corresponding end (13) of the compression screw (10).

Advantageously, the screw (4) is canulated to allow the passage of a guide wire (16′).

It is also described here a compression screw (10) characterized in that it comprises a device (1) as described.

Advantageously, the compression screw (10) and the device (1) are realized in a single piece.

Alternatively, advantageously, the device (1) is connectable in a detachable manner to the compression screw (10).

Last, it is also here described a stabilization device of a bone fracture, particularly a femoral fracture, comprising:

A plate (20) provided with a sliding channel (21);

A compression screw (10), having an end (13), and configured to result sliding into the channel (21) of the plate (20);

An element (1) in the shape of a pivot (1) connected to the screw (10) on the part of the end (13) and provided with at least one bevel (3).

In accordance with the invention, the bevel (3) sections the pivot askew according to a pre-determined β angle with respect to the longitudinal axis (14) in such a way as to at least reach the longitudinal axis (14) of the pivot (1), forming a flute-beak bevel, said pivot (1) being further short in such a way that, in use, said overall length of the pivot (1) in combination with said bevel (3) allow to realize the insertion of the channel (21) on the compression screw (10), through said pivot (1), also when the channel (21) is not initially arranged in axis with the screw (10).

### BRIEF DESCRIPTION OF DRAWINGS

Further characteristics and advantages of the present device, according to the invention, will result clearer with the description that follows of some preferred embodiments, made to illustrate but not to limit, with reference to the annexed drawings, wherein:

FIG. 1 shows an axonometric view of the compression screw and the plate in accordance with the state of the art;

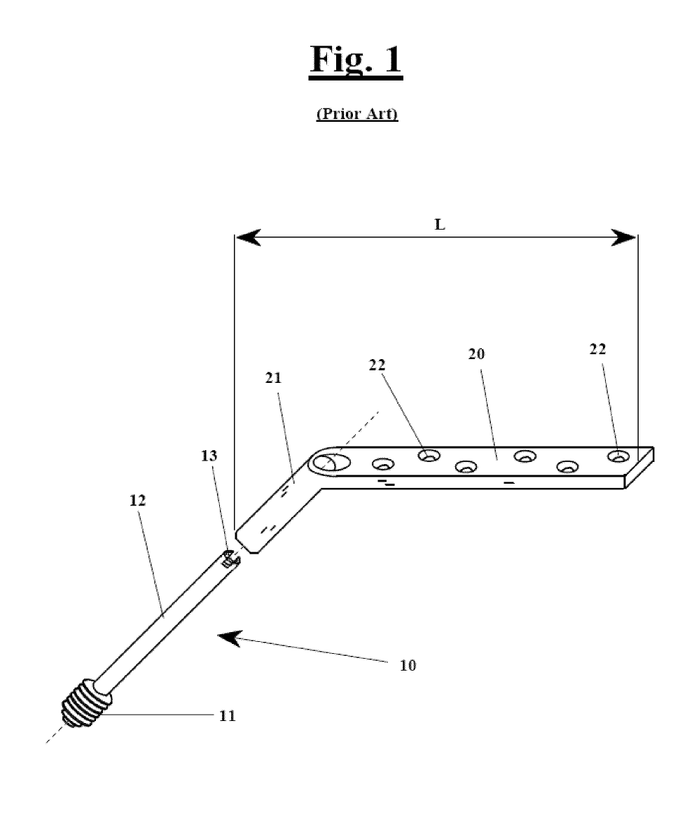

FIG. 2 shows, always in accordance with the state of the art, a schematic view of insertion of the screw in the bone and the sliding of the plate 20 on the rod of guide 30;

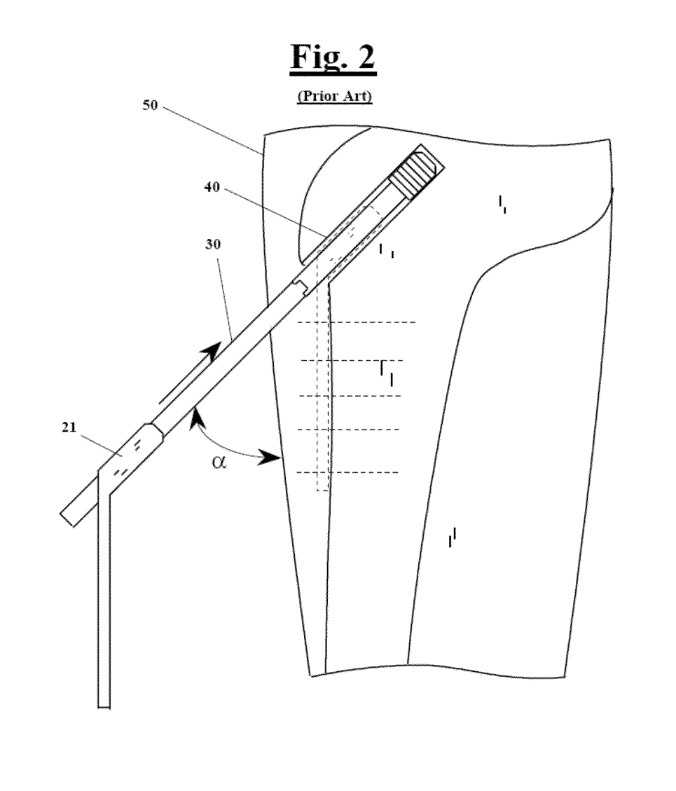

FIG. 3 shows an axonometric view of the compression screw and of the device 1;

FIG. 4 shows two different lateral views of the device 1, highlighting the bevel 3;

FIG. 5 shows a section of the device 1 highlighting the attachment means for connecting the device itself to the compression screw;

FIG. 6 shows a coupling between compression screw and device 1;

FIG. 7 shows schematically in an axonometric view the pivot 1 and the screw 10 from the part 13 in which the coupling takes place;

FIG. 8 shows three devices in accordance with the invention of different measures and of which one of them (the longest one) equipped with its screw 4 for coupling it to the compression screw 10 on the part of the end 13;

FIG. 9 shows in a frontal view and in section a possible realization geometry of the flute-beak pivot 1;

FIGS. 10 and 11 show two phases of insertion in the thigh of the plate with the help of said device;

FIGS. 12 and 13 further show two subsequent phases of application of the plate with the help of the device 1.

### DESCRIPTION OF SOME PREFERRED EMBODIMENTS

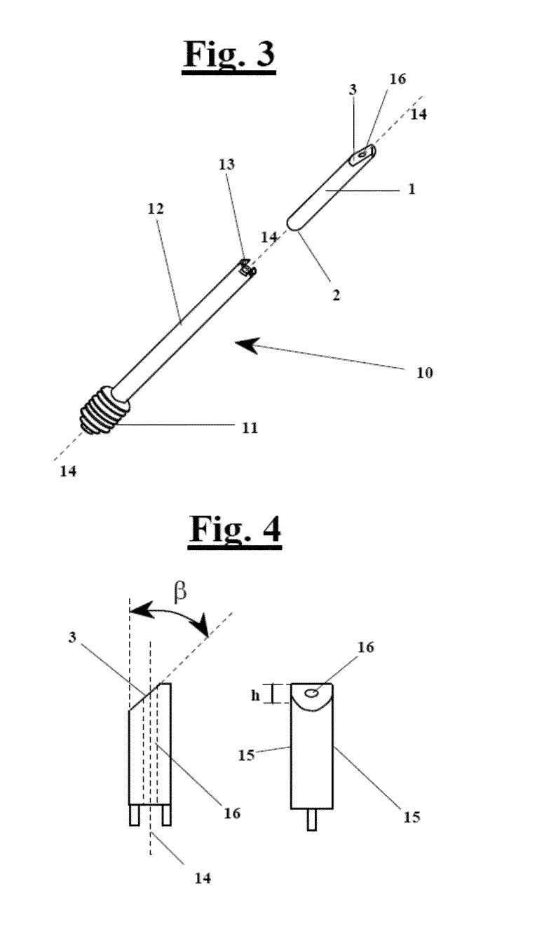

FIG. 3 shows, in an axonometric view, a schematization of the insertion device 1 for a fixing plate 20 in accordance with the invention.

The device 1 comprises a generally long-shaped element, for example a cylindrical pivot 1, configured in such a way as to be connected stably to the compression screw 10 on the part of its attachment 13.

The pivot can be realized in any type of bio-compatible material and has a length generally comprised between the 0.5 and 3 cm (preferably between 0.5 cm up to 2 cm), in such a way as to substantially exit by a millimetre, or something more, from the bone surface itself when, in use, it results applied to the screw which, in turn, is engaged in the bone tunnel. The range of length is therefore very inferior if compared with the overall length of about 15 centimetres relative to the rod 30 described in the state of the art. In this way, while the traditional rod 30 exits of various centimetres out of the thigh, said device 1 is kept below the muscular layer.

In accordance with the invention, the pivot 1 includes one of its ends 2 arranged to be connected stably, and in a removable manner, to the screw 10 on the part of its attachment 13 and an opposite end 3 which is bevelled.

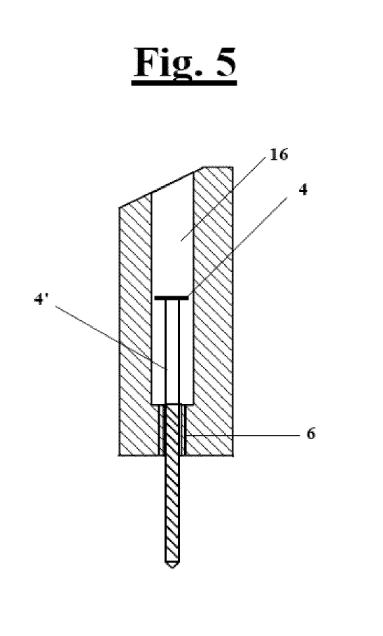

The β angle of the bevel (angle comprised between the longitudinal axis 14 of the pivot and the surface 3—see FIG. 4), can include a variable range of angles comprised between 20° and 80°, and preferably between 30° grades and 60° grades. The β angle of the bevel, as shown in FIG. 4, is measured with respect to the longitudinal axis 14 of the device 1. The height (h) of the bevel depends on the pre-chosen inclination and of the diameter of the device 1. In this regard, the pre-chosen diameters can be variable but are anyway equal or inferior to the overall diameter of the screw 10 to which the device is applied.

The bevel is such as to pass obliquely through the thickness of the pivot at least until reaching the longitudinal axis 14, in such a way as to form a flute-beak shaped surface.

As shown in FIG. 4 again, the cylindrical pivot 1 includes two sides 15 milled with a milling 15 configured in such a way that, in use, such millings 15 result an extension of the relative millings of the screw to which the device is applied. In this way, the pivot obliges a preventive insertion of the channel with an arrangement of the millings in line with those of the screw. In that way, any risk of block is avoided, once the channel reaches the screw, due to a lacking alignment of the internal milling of the channel 21 with the external one of the compression screw 10.

It is also clear that, even if the device 1 generally has the sides milled through two opposed millings, it could still have any number of millings on the basis of the millings present on the screw 10 to which it is coupled and therefore to those of the channel.

As shown in the two lateral views of FIG. 4, the device 1 is canulated, that is axially drilled through the formation of a channel 16 to allow at least the passage of the guide wire (internal dotted line). FIG. 4 shows the hole of entrance to the axial internal channel 16.

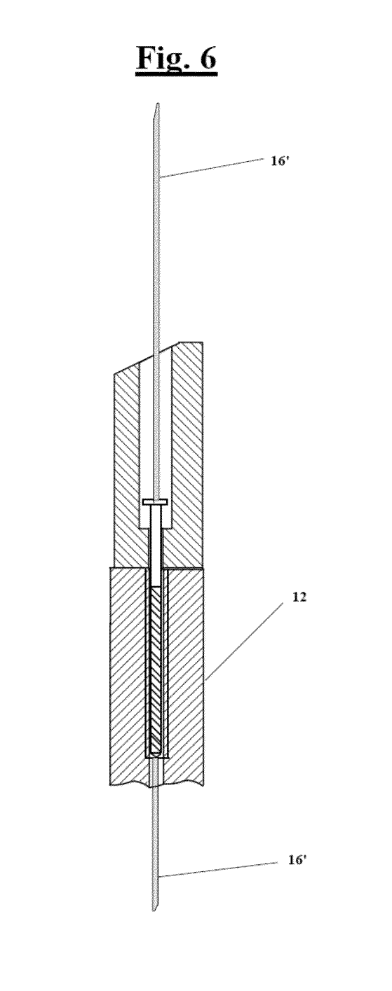

As shown schematically in the section of FIG. 5, appropriate fixing means (4, 16, 6) can be included to connect stably the pivot to the end 13 of the compression screw 10.

In a possible embodiment, such means can take advantage of the axial channel 16 of the pivot 1 and include a screw 4 that is arranged into the axial channel 16 itself. As shown in FIG. 5, the screw is threaded, preferably partially threaded (shank screw), in such a way as to be able to set on an equivalent screw thread 6 obtained on the final part of the channel 16. In this way, the screw 4 can be made integral to the channel 16 of the pivot.

The screw thread 6, that engages with the threaded final part of the screw 4, serves to avoid that the screw, once inserted into the 16 and exceeded the screw thread, can exit accidentally from the channel. In fact, the screw screws up to when the non threaded part (of a smaller diameter) slides freely into the portion of threaded channel 16. In this regard, the non threaded part 4′ has preferably a length greater with respect to the threaded portion 6 of the channel 16. In this way, the screw can be preventively arranged into the channel 16, screwing it up to when the screw thread of the screw 4 exceeds the screw thread 6 and, as a consequence, the part 4′ slides into the threaded channel 6 of a quantity equivalent to the excess of length between said part 4′ and the threaded part 6. In such an embodiment, not only is there the risk that the screw exits or gets lost, but also, at the same time, the backlash generated by said excess of length of the portion 4′ of non threaded screw with respect to the length of the portion 6 allows the surgeon, calmly, to easily apply only afterwards the pivot 1 to the end 13 of the compression screw 10 through the screw 4 preventively inserted into the channel 16.

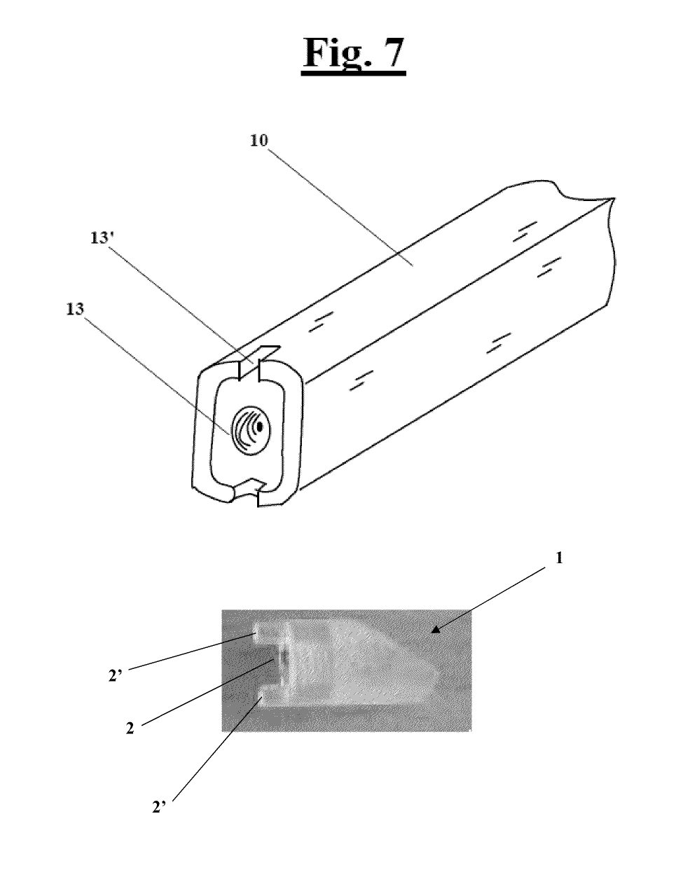

Through the screw 4, by screwing completely in the threaded channel of the end 13 of the compression screw 10, it is possible to apply preventively the pivot 1 to the compression screw 10 in such a way that the screw 10 results preventively equipped with such a device 1 at the time of the surgical engagement and can be removed once the plate has been inserted (see FIG. 6).

In particular, an attachment of the compression screw can be included comprising a threaded hole in such a way that the part of the threaded screw 4 exceeding can exit completely from the seat 6 to set on the thread of the compression screw. The pivot can therefore be overlapped to the compression screw, realizing a stable connection by simply screwing the screw 4 into the attachment 13 (see FIG. 6).

Naturally, also the internal screw 4 is canulated to allow the passage of the guide wire 16′. To that aim, FIG. 6 schematizes the device mounted on the screw 10 through the fixing screw 4 which is canulated to allow the passage of the guide wire 16′.

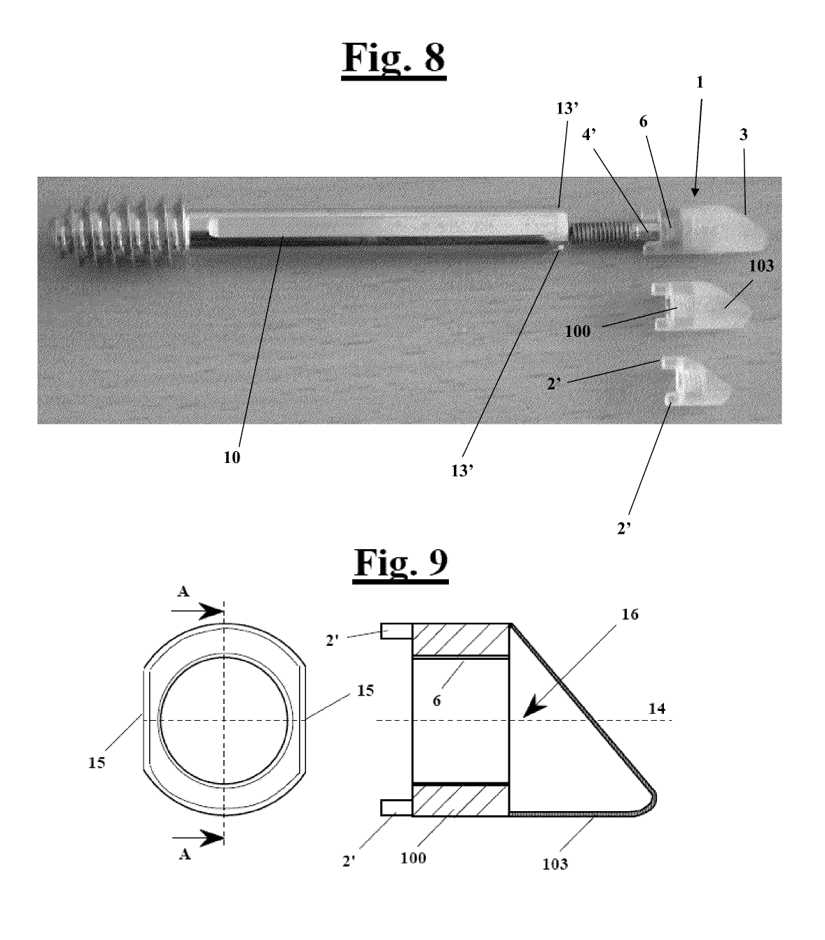

As schematically shown in FIG. 7, it is also clear that the base 2 of the pivot 1 is configured on the basis of the form of the base 13 of the screw 10 to which it is coupled in such a way as to be coupled to it in a perfect manner without relative rotations and with the millings that coincide so that the milling on the pivot is a continuation of the milling on the body of the compression screw 10. It is in fact preferable that, in the case of screw 10 milled on the sides, a milled pivot is used whose millings coincide with those of the screw 10 when the pivot is applied on the screw.

FIG. 7 also highlights to that aim two or more teeth 2′ belonging to the pivot and destined to engage each one of them into a complementary seat 13′ of the compression screw 10. The screwing of the screw 4 fixes the position, while the coupling through said teeth (2′; 13′) avoids the relative rotations and obliges to an only possible positioning that guarantees the continuity of the millings.

Otherwise, nevertheless, nothing would impede the use of a cylindrical pivot lacking millings even if, in this case, the functioning would not be optimal because the surgeon would have to find the correct rotation of the channel with respect to the screw 10 once the insertion of the pivot 1 into the channel 21 has taken place.

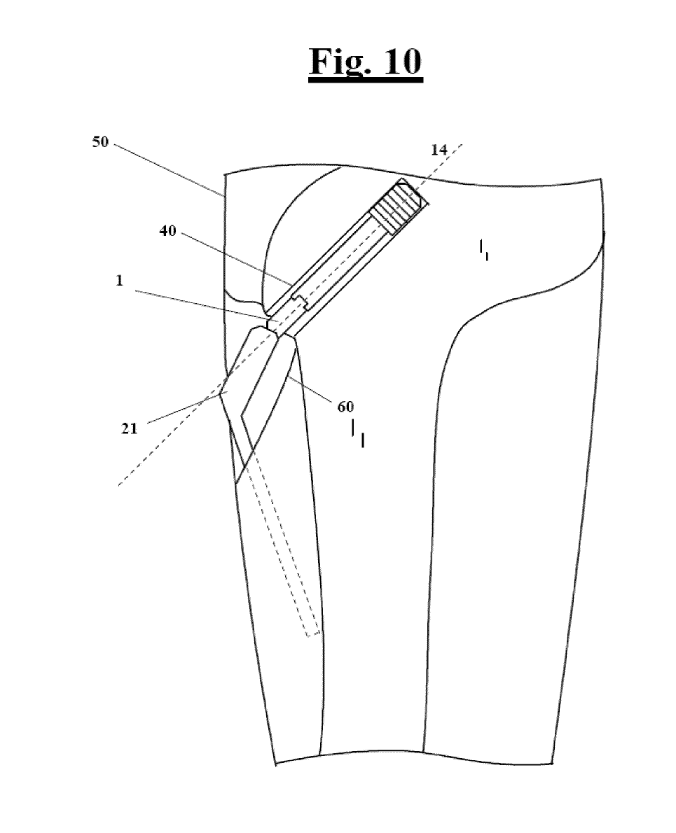

FIG. 8, as a way of example, shows a compression screw 10 drawn near in axis to a pivot 1, as described, and equipped with its screw 4. The figure highlights the teeth 2′ in axis with the seats 13′ that guarantee a coupling with continuity of milling and the non threaded portion 4′ of the screw 4 which exits from the final threaded portion 6 of the channel 16. FIG. 8 again, just as a way of example, shows three types of pivot 1 of different lengths (in this case 1 cm, 1.5 cm and 2 cm).

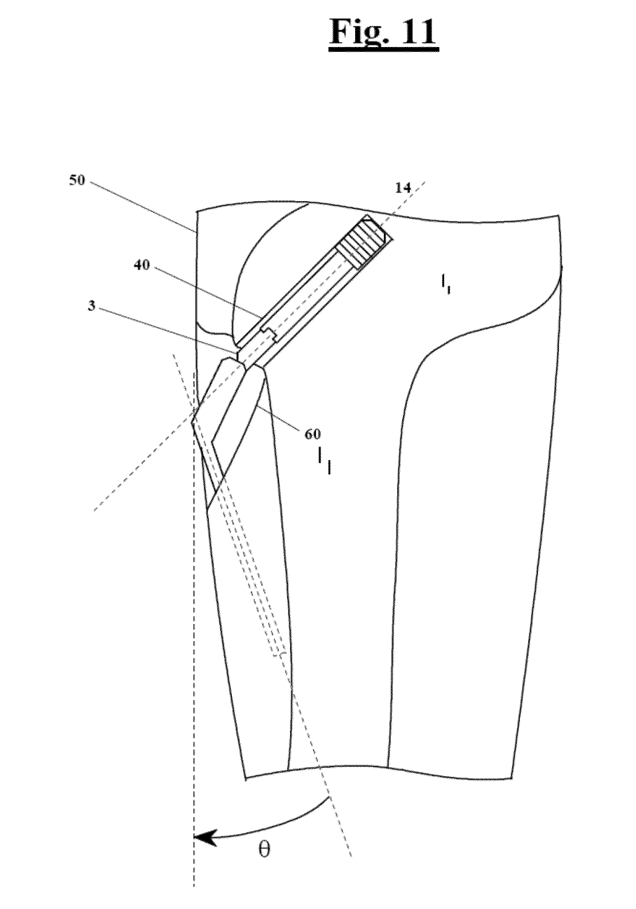

As shown in FIG. 8 and in FIG. 9, preferably but not necessarily, the pivot 1 can be formed by a central body 100, axially hollow and threaded, to form said portion 6, while the remaining part of the channel 16 is obtained through the application of a portion 103 conformed in the shape of a flute beak and of a greater diameter with respect to the portion 6 and non threaded (the active part of the device 1).

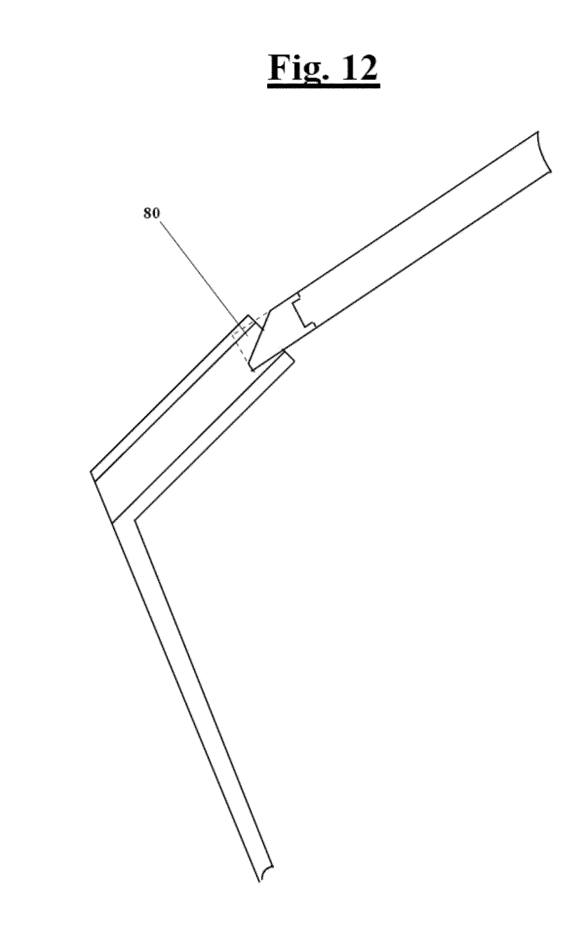

FIG. 10 schematizes a phase of application of the plate 20 to the compression screw 10 with the help of pivot 1 as described. The surgeon, not being anymore present a long rod that obliges to the direction of application, can now realize a small incision 60 precisely in correspondence of the pivot 1. the figure in question thus shows an opened incision to highlight the pivot 1 exiting from the channel 40 of some millimetres, anyway in such a way as to result substantially under the muscle. The necessary length of the incision 60 is therefore the one sufficient to allow an insertion of the plate from the part opposite to the channel 21 and its sliding under the muscle up to when the same reaches in position with the channel 11 close to the bevel 3.

At this point, as shown in FIG. 11, the surgeon can easily insert the channel on the compression screw 10 through the device 1. This is made possible by inserting the end of the bevel into the channel, making the milling of pivot 1 coincide with the one of the channel. Thanks to the bevel, it is now possible to rotate slightly the plate towards the bevel itself with a slight pressure, in such a way that the bevel is inserted completely into the channel (θ direction of rotation of FIG. 11), placing the channel in axis to the screw. The presence of the bevel therefore allows such a rotation and, for this reason, the angle of bevel is pre-chosen in such a way as to allow an angle rotation of the plate up to when the channel does not result in axis with the screw so that the channel can then normally slide along the screw.

It is thus evident that the angles of bevel can vary within the ranges indicated above on the basis of the internal diameter of the channel 21 and of the angle of positioning defined by the axis of the channel itself with respect to the plate. In any case, the pre-chosen angles of bevel are such as to allow a rotation, once the bevel has been inserted into the channel, that brings the channel in axis with the screw.

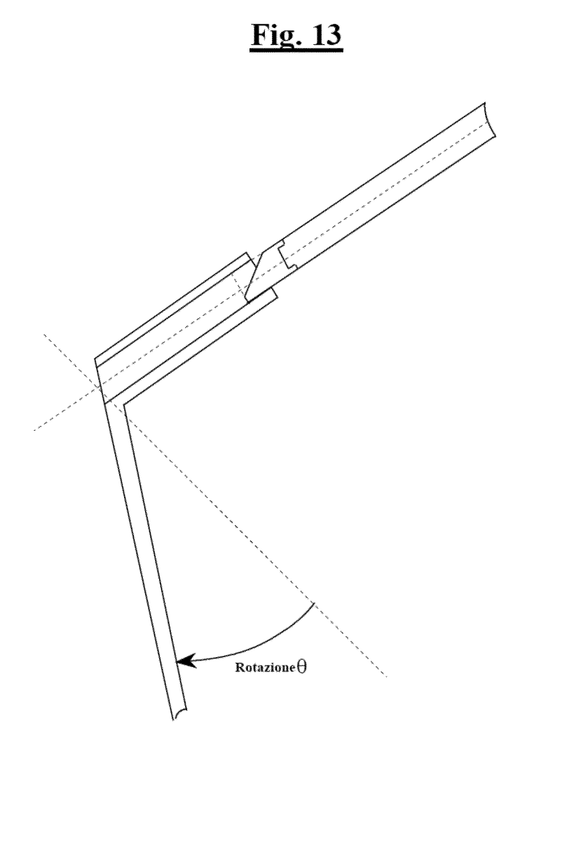

FIG. 12 and FIG. 13 schematically show a detail of insertion to highlight the functionality of the bevel. In particular, FIG. 12 shows, with a thin dotted line, the part of material 80 removed to realize the flute-beak bevel 3 with the aim of better highlighting how such a material 80, if present, would not allow the insertion since it would stop against the edges of the channel when not in axis. FIG. 13 shows the completed rotation for bringing in axis the channel to the device and realizable precisely thanks to the removal of the part of material 80 that has realized the bevel.

Once the plate has been inserted, the normal unscrewing of the screw 4 can take place in such a way as to remove the pivot 1 from the compression screw.

Although a solution in which the device 1 is applied in a removable manner to the compression screw 10 has been described, it is anyway predictable also the realization of a compression screw that includes an integrated bevel as described. In this regard, it would be enough to realize a slightly longer standard screw in such a way as to exit from the bone plane, and provided with said bevel.

Moreover, the bevel does not have to be necessarily one but also two or more bevels can be included on the apex, appropriately combined in such a way as to allow the insertion of the channel as described.

1. A device for allowing an application of a fixing plate, provided with a sliding channel, on a compression screw in a bone fracture, the device comprising:
a pivot-shaped element provided with a fixing means for applying said pivot-shaped element to an end of said compression screw, said pivot-shaped element is provided, on an opposite side destined to connect with the end, with at least one bevel, said at least one bevel sections the pivot-shaped element askew according to a pre-determined angle with respect to a longitudinal axis of said pivot-shaped element in such a way as to at least reach the longitudinal axis of the pivot-shaped element, forming a flute-beak bevel, said pivot-shaped element being further short in such a way that in use an overall length of the pivot-shaped element in combination with said at least one bevel allow to realize an insertion of the sliding channel on the compression screw, through said pivot-shaped element, also when the sliding channel is not initially arranged in axis with the compression screw.

2. A device according to claim 1, wherein said at least one bevel sections the pivot-shaped element askew through an entire thickness of said pivot-shaped element.

3. A device according to claim 1, wherein said pivot-shaped element comprises two millings, opposed in such a way as to result insertable into a relative milled seat of the sliding channel of the plate without relative rotation between the plate and said pivot-shaped element.

4. A device according to claim 1, wherein said pivot-shaped element comprises at least a pair of teeth for engaging each one of said teeth into a respective complementary seat of the end of the compression screw in such a way as to oblige to realize an application of the pivot-shaped element to the compression screw so that there is a continuity in millings present on the compression screw and on the pivot-shaped element.

5. A device according to claim 1, wherein said angle is comprised within a range between 20° and 80° with respect to the longitudinal axis of the pivot-shaped element, and preferably within a range between 30° and 60°.

6. A device according to claim 1, wherein said pivot-shaped element has an overall length comprised between about 0.5 cm and about 3 cm, and preferably between about 0.5 cm and about 2 cm, in such a way that, in use, said pivot-shaped element remains under muscle.

7. A device according to claim 1, wherein said pivot-shaped element is axially holed and forms an axial channel.

8. A device according to claim 1, wherein said pivot-shaped element is substantially cylindrical.

9. A device according to claim 1, wherein said fixing means comprises:
a screw thread placed in a final part of an axial channel of the pivot-shaped element;
a screw placed into the axial channel and having at least one threaded final part in such a way as to be able to screw the screw to the thread of the axial channel, exiting from said axial channel at least in part to be able to engage into a corresponding screw thread arranged in axis at the end of the compression screw.

10. A device according to claim 9, wherein the screw includes a first part non threaded, and a final part, threaded, a diameter of the non threaded part being inferior to a diameter of a seat of the pivot-shaped element into which the screw is inserted and a length of the non threaded part being greater than a length of the threaded part in such a way that, when the threaded part of the screw has exceeded the screw thread of the axial channel, exiting from the pivot-shaped element, the non threaded part results sliding with respect to the threaded part of the axial channel of a quantity equivalent to an excess of length between the non threaded part and the length of the threaded part in such a way as to allow to continue screwing of the screw in the corresponding end of the compression screw.

11. A device according to claim 9, wherein the screw is canulated to allow passage of a guide wire.

12. A compression screw, comprising:
a device for allowing an application of a fixing plate, provided with a sliding channel, on a compression screw structure in a bone fracture, the device comprising a pivot-shaped element provided with a fixing means for applying said pivot-shaped element to an end of the compression screw structure, wherein said pivot-shaped element is provided, on an opposite side destined to connect with the end, with at least one bevel, said at least one bevel sections the pivot-shaped element askew according to a pre-determined angle with respect to a longitudinal axis of said pivot-shaped element in such a way as to at least reach the longitudinal axis of the pivot-shaped element, forming a flute-beak bevel, said pivot-shaped element being further short in such a way that in use an overall length of the pivot-shaped element in combination with said at least one bevel allow to realize an insertion of the sliding channel on the compression screw structure, through said pivot-shaped element, when the sliding channel is not initially arranged in axis with the compression screw structure.

13. A compression screw according to claim 12, wherein the compression screw and said device are realized in a single piece.

14. A compression screw according to claim 12, wherein the device is connectable in a detachable manner to the compression screw.

15. A stabilization device of a bone fracture, particularly a femoral fracture, comprising:
a plate provided with a sliding channel;
a compression screw having an end, and said compression screw being configured to result sliding into the sliding channel of the plate;
an element in a shape of a pivot connected to the compression screw on a part of the end and provided with at least one bevel, wherein said at least one bevel sections the pivot askew according to a pre-determined angle with respect to a longitudinal axis of the pivot in such a way as to at least reach the longitudinal axis of the pivot, forming a flute-beak bevel, said pivot being further short in such a way that, in use, an overall length of the pivot in combination with said at least one bevel allow to realize insertion of the sliding channel on the compression screw, through said pivot, also when the sliding channel is not initially arranged in axis with the compression screw.

16. A device according to claim 7, wherein said fixing means comprises:
a screw thread placed in a final part of the axial channel of the pivot-shaped element;
a screw placed into the axial channel and having at least one threaded final part in such a way as to be able to screw the screw to the thread of the axial channel, exiting from said axial channel at least in part to be able to engage into a corresponding screw thread arranged in axis at the end of the compression screw.
